(19) United States
(12) Patent Application Publication
(10) Pub. No.: US 2002/0001615 A1
(43) Pub. Date: Jan. 3, 2002

(54) Combined interferon alfa and lipsosmal-encapsulated all-trans retinoic acid, including preparation and use

(75) Inventor: David Nanus, New York, NY (US)

Correspondence Address: Thomas M. Saunders, Lorusso & Loud, 440 Commercial Street, Boston, MA, 02109, US

(73) Assignee: Aronex Pharmaceuticals, Inc.

(21) Appl. No.: 09/811,346

(22) Filed: Mar. 16, 2001

Related U.S. Application Data
(60) Provisional application No. 60/193,565, filed on Mar. 31, 2000.

(51) Int. Cl. A61K 9/127; A61K 38/21; A61K 31/203

(52) U.S. Cl. 424/450; 424/85.7; 514/559

(57) ABSTRACT

Alfa interferon (α-IFN or alpha-interferon) and liposomal all-trans retinoic acid is useful in cancer treatment with particular reference to renal cancer. Optionally, a regimen of α-interferon from about 3 to about 5 million units sc daily and liposomal all-trans retinoic acid at from about 15 mg/m² to about 90 mg/m², or about 140 mg/m², or about 300 mg/m² or more. Dosage periodicity of about three times per week for both drugs in about 8 week cycles is useful.

### CROSS REFERENCE TO RELATED APPLICATIONS

This application claims priority to Provisional application No. 60/193,565 filed Mar. 31, 2000.

### FIELD OF THE INVENTION

Alfa interferon (α-IFN or alpha-interferon) and liposomal all-trans retinoic acid is useful in cancer treatment with particular reference to renal cancer. Optionally, a regimen of α-interferon from about 3 to about 5 million units sc daily and liposomal all-trans retinoic acid (e.g., ATRAGEN®, Aronex Pharmaceuticals, The Woodlands, Tex.) at a dose from about 15 mg/m² to about 90 mg/m², or about 140 mg/m², or about 300 mg/m² or more. Dosage periodicity of about five times per week for both drugs in about 8 week cycles is useful. In some instances interferon is dosed more often including every other day and daily.

### BACKGROUND OF THE INVENTION

The incidence of renal cell carcinoma is estimated to be approximately 30,000 new cases annually, with a death rate of 10,000 patients per year (1). At the time of diagnosis approximately fifty percent of patients have disease localized to the kidney, thirty percent of patients have distant metastases, and the remaining twenty percent of patients have locally advanced disease (2). Currently, surgical resection of all discernible disease is the only potentially curative therapy. For patients with stage I or II disease, the five year survival ranges from 45 to 85%, while for patients with stage III disease the five year survival ranges from 15 to 35% (2). Occasionally, selected patients with stage IV disease have prolonged disease free survival after resection of solitary metastases.

For those patients with surgically unresectable disease, therapeutic options include chemotherapy, hormonal therapy and immunotherapy. Unfortunately, all of these therapies are relatively unsuccessful. Hormonal therapy has little or no therapeutic effect (3). Similarly, available chemotherapy has been generally ineffective. More than 40 drugs have been investigated, but none achieved a response proportion greater than 15-20% alone or in combination (4). The potential therapeutic benefit of biologic response modifiers like interferons (IFN) have been studied in RCC (5). Queseda, et al, first reported the clinical efficacy of human leukocyte IFN in metastatic RCC (6). Subsequently, numerous clinical trials with various subtypes of IFN including purified human lymphoblastoid interferon-alpha and purified recombinant interferon-alpha 2a and 2b have been performed. In these trials, the proportion of patients experiencing major responses is only about 15% (and a range of 5-29%), with a median duration of response ranging from three to 16 months (5; 7). In a review of 18 trials of renal carcinoma treated with interferon-alpha, Krown et al found no significant difference in response based on type or schedule of drug (7). There was, however, evidence that moderate doses of interferon-alpha produced superior response rates when compared to either low or high doses. Thus, the overwhelming majority of patients with RCC are unresponsive to the antitumor effects of IFN given as a single agent (8; 9).

Other clinical trials have investigated the efficacy of other biological response modifiers alone or in combination with IFN a in the treatment of patients with metastatic RCC (10; 11). Interleukin-2 (IL-2), with or without lymphokine-activated killer (LAK) cells, has been extensively studied. Although initial clinical trials reported significant numbers of major clinical responses with IL-2, this was associated with significant toxicity and few patients have shown long term clinical benefit (12; 13). The addition of interleukin-2 (IL-2) to IFN resulted in a higher number of clinical responses in patients with advanced RCC in one study (14), however, this was not observed in subsequent trials (15; 16). Overall, the data suggest that, similar to IFN a, the proportion of patients experiencing significant responses with IL-2 based therapy is approximately 15% (17). It is clear that the need exists for more effective therapy for patients with advanced renal cancer.

A phase II trial of Interferon alfa-2a and free (non-liposomal) 13-cis-retinoic acid (CRA) was conducted at Memorial Sloan-Kettering Cancer Center (MSKCC) in patients with advanced renal cell carcinoma (RCC). IFN was given daily; starting at 3 million units (MU) and the dose was escalated every seven days from 3 to 6 to 9 MU. The CRA was given daily at a dose of 1 mg/kg/day. Thirteen (30%) of 43 evaluable patients achieved a major response (three complete, ten partial) (34). In addition to lung and nodal metastases, responding sites included bone metastases and renal primary tumors.

Other trials have also reported using a combination of 13-cis retinoic acid and IFN (36; 37). In one study examining the pharmacokinetics of free all-trans retinoic acid (ATRA) in patients with renal cancer concomitantly treated with IFN, peak levels of atra in the serum declined after three months on therapy (38).

### SUMMARY OF THE INVENTION

This invention comprises a method of inhibiting the growth of cancer cells, and particularly renal cancer cells, comprising exposing cancerous cells to a therapeutically effective amount of a composition which comprises at least one interferon and a retinoid, wherein said retinoid is associated with lipid carrier particles. Particular note is made of the method the retinoid is retinoic acid, such as all-trans retinoic acid.

In some embodiments of the method the lipid carrier particles comprise all-trans retinoic acid, lipid, and a triglyceride and the molar ratio of retinoid to lipid is at least about 15:85, where the triglyceride is at least about 15% by weight of the composition, and where the composition is stable in an aqueous environment. In specific embodiment the method of comprises administering said retinoid composition in doses administered over a period of at least one-half hour, and, optionally, administering said retinoid composition at a frequency of about every other day or less frequent.

In another embodiment this invention comprises a method of inhibiting the growth of cancer cells comprising exposing cancerous cells to a therapeutically effective amount of a composition which comprises at least one interferon and further co-timely exposing of said cancerous cells to a therapeutically effective amount of a retinoid, wherein said retinoid is associated with lipid carrier particles.

A composition of the present invention comprises a therapeutic treatment kit for the treatment of cancer comprising interferon, retinoid and instructional materials for the combined use of said retinoid and interferon. In some instances instructional materials include such information as dosage, indication, and contraindication and storage parameters.

### DETAILED DESCRIPTION OF THE INVENTION

A. “Exposing” as used in relation to cancerous cells shall mean in vivo and further include extra corporeal as will as in vitro applications. In vitro applications are particularly useful in diagnostic and screening applications of the present invention.

B. Cancer shall be broadly understood to mean an abnormal uncontrolled growth of tissue that has potential to spread to distant sites of the body. In particular, cancer shall include renal cell carcinoma including chromophobe cell renal carcinoma and further granular/eosinophilic variants of these tumors and renal oncocytoma, renal leiomyosarcoma. Particular note is made of head, neck, and breast cancer. Head, neck, and breast cancer are often found to have reduced retinoid levels. In specific instances tumor cells presenting with low retinoid levels exhibit enhanced therapeutic response to the instant therapy.

C. “Therapeutically effective amount” is defined independently for each drug. As to L-ATRA a therapeutically effective amount shall mean about 15-300 mg/m² and particularly 90 mg/m².

As to interferon alfa a therapeutically effective amount shall mean from about 1 to about 25 million IU and particularly 3-5 million IU.

It is anticipated that interferons alfa, beta, gamma, and omega are administered in similar doses. Doses are generally adjusted to at or below the maximum tolerated dose (MTD). Signs indicative of interferon toxicity are noted to be as to hematologic toxicity, anemia, thrombocytopenia, leukopenia: as to gastrointestinal toxicity, diarrhea, dyspepsia, dysphagia, N/V, abdominal pain; as to liver toxicity increases in bilirubin, alk phos and LFTs; as to kidney and bladder, microscopic hematuria, pyuria, azotemia, proteinuria, acute renal failure, nephrotic syndrome, glycosuria, albuminuria; as to pulmonary, orthopnea, dyspnea, bronchospasm, coughing, pulmonary edema, ARDS; as to cardiac toxicity syncope, MI, SVT, bradycardia, tachycardia, dizziness, hyptoension, hypertension. Neurological toxicity are confusion, tremors, numbness, paresthesia, inability to concentrate, somnolence, hallucinations, encephalopathy, seizure, coma, psychomotor retardation, memory dysfunction, dry mouth, sweating, personality disorder, agitation, neuropathy, depression, anxiety, aphasia, retinal infarction with vision loss, eye pain, hemianopsis, taste change, headache, syncope, insomnia. Dermal toxicity of skin rash, urticaria, epidermal necrosis, maculopapular rash is noted. Metabolic toxicity manifests as hyperglycemia. In addition coagulation is monitored for increase in PT/PTT. Also the presence of phyarngitis, alopecia, fatigue, malaise, anorexia, weight loss, fever, chills, myalgia, arthralgia, cyanosis are potential toxic responses to interferon.

Liposomal ATRA at toxic doses displays hematologic thrombocytopenia. In addition gastrointestinal toxicity of N/V and mucositis. Liver toxicity increase alk phos and LDH. Neurologic toxicity results in emotional changes, and headache. Dermal toxicity is noted in dry skin, dermatitis. Also, metabolic changes are found in an increase in triglycerides levels in the blood. Toxicity is also determined by alopecia, anorexia, dry eyes, cheilitis, epistaxis, joint pain, fatigue, pruritus, and conjunctivitis.

The foregoing not withstanding, a supervising clinician will understand that initial myelosupression is a favorable sign in the treatment of leukemias.

Without being bound by any particular theory it is believed that retinoid effects are mediated through retinoic acid nuclear receptors (RARs) which are members of the steroid receptor superfamily of ligand-dependent transcriptional factors (25). Two distinct retinoid nuclear receptor systems exist, the RARs (RAR-a, -b, -g ) and the RXRs (RXR-a, -b, -g ) (26). The RARs and RXRs can heterodimerize following RA binding, and transcriptionally activate or repress other genes which mediate the growth and differentiation effects of RA (26; 27).

D. “Interferon” shall be broadly understood to mean any of several glycoproteins that help the body fight off viral infections. Particular note is made of interferons alfa (or alpha), beta, and gamma. Interferon alpha is the main type of interferon produced by the white blood cells

Particular reference is made to interferon alfa-2b, recombinant, (Intron A, Schering), and interferon alfa 2a (Roferon, Hofman LaRoche).

E. “Retinoid” shall be broadly understood to mean the natural and synthetic derivatives of vitamin A. Isotretinoin (13 cis-retinoic acid) and tretinoin (all trans retinoic acid) represent the two naturally occurring isomers of retinoic acid (18).

F. Lipid Carrier particle shall be expansively understood to mean all lipid-drug particulates. Reference also is made to U.S. Pat. No. 5,811,119, “Formulation and Use of Carotenoids in Treatment of Cancer” to Mehta et al. Reference is further made to U.S. Pat. No. 4,610,868 to Fountain. Fountain is a patent which describes amorphous lipid particles, with particular reference to Fountain col. 7, lines 1-17. Lipid carrier particles is a term known in the art defining structures in addition to liposomes.

Particular reference is made to liposomal ATRA. In one embodiment. Liposomal ATRA or liposomal tretinoin (also known as liposomal ATRA Tretinoin^{LF} or ATRAGEN®) is provide by Aronex Pharmaceuticals, Inc (The Woodlands, Tex.). Without being bound by any particular theory, the liposomal delivery system improves the activity of the tretinoin by altering its pharmacological profile, such as changing the drug's pharmacokinetics and issue distribution. Once injected into the bloodstream, liposomes are quickly cleared by the reticuloendothelial system (RES) cells which include the liver and spleen and, most importantly, the hematopoietic tissues from which the malignant cells are seeded. Minimal liposomal uptake occurs in tissues with continuous, non-fenestrated capillaries such as muscle and nervous tissue.

Another beneficial difference is that the lipid formulation bypasses the clearance mechanism that evolves in the livers of patients treated with the oral formulation. In addition, toxicities associated with oral doses of tretinoin are reduced in some cases because liposome encapsulation of tretinoin decreases direct exposure of the tretinoin during circulation to levels below the orally administered toxic dose. The latter allows greater total exposure of the drug on initial dose accompanied by slower clearance of the tretinoin. This is also understood to be an avoidance of ATRA resistance.

G. “Co-timely” as to drug administration shall mean administration of interferon while L-ATRA is present in a therapeutically effective amount or the reverse. It is to be understood that in some instances this will require sequential administration. In some instances, multiple routes of administration will be employed such as intravenous or subcutaneous injection of an alfa interferon, while a L-ATRA is administered i.v. prior to or subsequent to such interferon administration.

Treatment is usefully employs liposomal ATRA in the form of ATRAGEN®. A vial of lyophilized ATRAGEN® is reconstituted with 50 ml of 0.9% sodium chloride for injection, USP, to provide a 2 mg per ml of liposomal suspension requiring no further dilution steps. The vial is then shaken vigorously for one minute. This forms a dispersion of ATRAGEN® liposomes. Several minutes is then for the foaming of reconstituted product to subside prior to transfer of the suspension. Due to the foaming of the reconstituted product, approximately 5-10 mL of the 50 mL of product may not be transferable. At this point, the reconstituted drug is aseptically transferred into an I.V. bag or bottle. Alternatively, properly cover the I.V. bag or bottle to sufficiently reduce light exposure during infusion (I.V. lines do not generally require coverage. As to interferon-alfa 2b, Inton A, (Schering Oncology)., this is available as a reconstituted solution for injection in 3, 5 and 10 million IU vials. Each vial contains 3 (or 5 or 10) million IU of Interferon alfa-2b, recombinant, dissolved in 0.5 ml (3 and 5 million unit vials) or 1 ml (10 million unit vials). Each 1 ml contains 7.5 mg sodium chloride, 1.8 mg sodium phosphate dibasic, 1.3 mg sodium phosphate monobasic, 0.1 mg edetate disodium, 0.1 mg polysorbate 80, and 1.5 mg m-cresol as preservative. Vials are stored in refrigerator (4° C.) prior to use and is stable for up to 7 days at 35° C. and at 30° C. for up to 14 days.

In some instances, interferon is administered s.c. Blood levels tend to peak at about 4 hours. For patient comfort, interferon is usefully administered in the evening so that a subject will be asleep during the more severe side-effects. Co-timely administration particular is noted to present ATRAGEN® concentrations to coincide with interferon peaks. In one embodiment, interferon is administered Monday through Friday and ATRAGEN® Monday, Wednesday and Friday.

### EXAMPLE 1

A 63 year old human male presented with metastatic renal cancer. Alfa interferon and ATRAGEN® were administered as follows:

Interferon at 5×10⁶ units s.c. daily Monday through Friday, and ATRAGEN® 15 mg/m² i.v., Monday, Wednesday and Friday. This treatment was provided in 8 week cycles resulting in regression of the cancer.

Relevant additional information is available in the following:

1. Parker, S. L., Tong, T., Bolden, S., and Wingo, P. A. Cancer statistics, 1997. CA—Cancer J Clin, 47: 5-27, 1997.

2. Motzer, R. J., Bander, N. H., and Nanus, D. M. Renal-cell carcinoma. N. Engl. J. Med., 335: 865-875, 1996.

3. Yagoda, A., Petrylak, D., and Thompson, S. Cytotoxic chemotherapy for advanced renal cell carcinoma. [Review]. Urologic Clinics of North America, 20: 303-321, 1993.

4. Motzer, R. J. and Vogeizang, N. J. Chemotherapy for renal cell carcinoma. In: D. Raghavan, H. I. Scher, S. A. Leibel and P. Lange (eds.), Principles and practice of genitourinary oncology, pp. 885-896, Philadelphia: Lippincott-Raven Publishers. 1997.

5. Buzaid, A. C. and Todd, M. B. Therapeutic options in renal cell carcinoma. Semin. Oncol., 16: 12-19, 1989.

6. Quesada, J. R., Swanson, D. A., Trindade, A., and Gutterman, J. U. Renal cell carcinoma: antitumor effects of leukocyte interferon. Cancer Res., 43: 940-947, 1983.

7. Krown, S. E. Interferon treatment of Renal Cell Carcinoma. Cancer, 59: 647-651, 1987.

8. Quesada, J. R. Role of interferons in the therapy of metastatic renal cell carcinoma. Urology, 34: 80-83, 1989.

9. Horoszewicz, J. S. and Murphy, G. P. An assessment of the current use of human interferons in therapy of urological cancers. Urology, 142: 1173-1180, 1989.

10. Quesada, J. R. Biologic Response Modifiers in the Therapy of Metastatic Renal Cell Carcinoma. Seminars in Oncology, 15: 396-407, 1988.

11. Haas, G. P., Hillman, G. G., Redman, B. G., and Pontes, J. E. Immunotherapy of renal cell carcinoma. CA-A Cancer J. Clinicians, 43: 177-187, 1993.

12. Kragel, A. H., Travis, W. D., Steis, R. G., Rosenberg, S. A., and Roberts, W. C. Myocarditis or acute myocardial infarction associated with interleukin-2 therapy for cancer. Cancer, 66: 1513-1516, 1990.

13. Rosenberg, S. A. Immunotherapy and gene therapy of cancer. Cancer Res., 51: 5074s-5079s, 1991.

14. Figlin, R. A., Belidegrun, A., Moldawer, N., Zeffren, J., and desertion, J. Concomittant administration of recombinant human interleukin-2 and recombinant interferon alfa-2a: An active outpatient regimen in metastatic renal cell carcinoma. J Clin Ankle., 10: 414-421, 1992.

15. Ilion, D. H., Motzer, R. J., Creation, R. G., Vogelzang, N. J., Bajorin, D. F., Scher, H. I., Nanus, D., OMoore, P., Marathias, K., and Bosl, G. J. A phase 11 trial of interleukin-2 and interferon alfa-2a in patients with advanced renal cell carcinoma. J Clin Ankle., 10: 11 24-1130, 1992.

16. Atkins, M. B., Sparano, J., Fisher, R. I., Weiss, G. R., Margolin, K. A., Fink, K. I., Rubinstein, L., Louie, A., Mier, J. W., Gucalp, R., Sosman, J. A., Boldt, D. H., Doroshow, J. H., Aronson, F. R., and Sznol, M. Randomized phase II trial of high-dose interleukin-2 either alone or in combination with interferon alfa-2 b in advanced renal cell carcinoma. J Clin Ankle., 11: 661-670, 1993.

17. Wirth, M. P. Immunotherapy for metastatic renal cell carcinoma. Urol. Clin. North. Am., 20: 283-295, 1993.

18. Lippman, S. M. and Meyskens, F. L. Jr. Vitamin A derivatives in the prevention and treatment of human cancer. J. Am. Coll. Nutr., 7: 269-284, 1988.

19. Smith, M. A., Parkinson, D. P., Cheson, B. D., and Friedman, M. A. Retinoids in cancer therapy. J Clin.Oncol., 10: 839-864, 1992.

20. Lippman, S. M. and Meyskens, F. L., Jr. Results of the use of vitamin A and retinoids in cutaneous malignancies. Pharmacol. Ther., 40: 107-122, 1989.

21. Kraemer, K. H., Di-Giovanna, J. J., Moshell, A. N., Tarone, R. E., and Peck, G. L. Prevention of skin cancer in xeroderma pigmentosum with the use of oral isotretinoin. N Engl. J. Med., 318: 1633-1637, 1988.

22. Hong, W. K., Endicott, J., and Itri, L. M. 13-cis-retinoic acid in the treatment of oral leukoplakia. N. Eng. J. Med., 315: 1501-1505, 1986.

23. Frankel, S. R., Eardley, A., Heller, G., Berman, E., Miller, W. H., Jr., Dmitrovsky, E, and Warrell, R. P., Jr. All-trans retinoic acid for acute promyelocytic leukemia. Results of the New York Study. Ann. Intern. Med., 120: 278-286, 1994.

24. Muindi, J., Frankel, S. R., Miller, W. H., Jr., Jakubowski, A., Scheinberg, D. A., Young, C. W., Dmitrovsky, E., and Warrell, R. P., Jr. Continuous treatment with all-trans retinoic acid causes a progressive reduction in plasma drug concentrations: implications for relapse and retinoid “resistance” in patients with acute promyelocytic leukemia [published erratum appears in Blood 1992 Aug. 1;80(3):855]. Blood, 79: 299-303, 1992.

25. Evans, R. The steroid and thyroid hormone receptor superfamily. Science, 240: 889-895, 1988.

26. Pemrick, S. M., Lucas, D. A., and Grippo, J. F. The retinoid receptors. [Review]. Leukemia, 8 Suppl 3: S1-10, 1994.

27. Chambon, P. The retinoid signaling pathway: molecular and genetic analyses. [Review]. Semin. Cell Biol, 5: 115-125, 1994.

28. Marth, C., Daxenbichler, G., and Dapunt, O. Synergistic antiproliferative effect of human recombinant interferons and retinoic acid in cultured breast cancer cells. J. Natl. Cancer Inst., 77: 1197-1197, 1986.

29. Frey, J. R., Peck, R., and Bollag, W. Antiproliferative activity of retinoids, interferon alpha and their combination in five human transformed cell lines. Cancer Letters, 57: 223-227, 1991.

30. Bollag, W. and Peck, R. Modulation of growth and differentiation by combined retinoids and cytokines in cancer. In: W. K. Hong and R. Lotan (eds.), Retinoids in oncology, pp. 89-108, New York: Marcel Dekker, Inc. 1993.

31. Arbaje, Y. M., Bittner, G., Yingling, J. M., Storer, B., and Schiller, J. H. Antiproliferative effects of interferons alpha and beta in combination with 5-fluorouracil, cisplatin, and cis- and trans-retinoic acid in three human lung carcinoma cell lines. J Interferon Res, 13: 25-32, 1993.

32. Lippman, S. M., Parkinson, D. R., Itri, L. M., Weber, R. S., Schantz, S. P., Ota, D. M., Schusterman, M. A., Krakoff, I. H., Gutterman, J. U., and Hong, W. K. 13-cis-retinoic acid and interferon alpha-2a: effective combination therapy for advanced squamous cell carcinoma of the skin. J. Natl. Cancer Inst., 84: 235-241, 1992.

33. Lippman, S. M., Kavanagh, J. J., Paredes-Espinoza, M., Delgadillo-Madrueno, F., Paredes-Casillas, P., Hong, W. K., Holdener, E., and Karakoff, I. H. 13-cis-retinoic acid plus interferon alpha-2a: highly active systemic therapy for squamous cell carcinoma of the cervix. Reports, 84: 241-245, 1992.

34. Motzer, R. J., Schwartz, P., Murray Law, T., Hoffman, A. D., Albino, A. P., Viamis, V., and Nanus, D. M. Antitumor effects of interferon alfa-2a and 13cis-retinoic acid in renal cell carcinoma: Results of a phase II trial and in vitro studies. J Clin Ankle., 13: 1950-1957, 1995.

35. Berg, W. J., Schwartz, L. H., Amsterdam, A., Mazumdar, M., Murray-Law, T., Vlamis, V., Nanus, D. M., and Motzer, R. J. Clinical studies with 13-cis-retinoic acid in patients with advanced renal cell carcinoma. Invest. New Drugs 15(4):353-5 (1997).

36. Buer, J., Probst, M., Ganser, A., and Atzpodien, J. Response to 13-cis-retinoic acid plus interferon alfa-2a in two patients with therapy-refractory advanced renal cell carcinoma [letter]. Journal of Clinical Oncology, 13: 2679-2680, 1995.

37. Atzpodien, J., Kirchner, H., Duensing, S., Lopez Hanninen, E., Franzke, A., Buer, J., Probst, M., Anton, P., and Poliwoda, H. Biochemotherapy of advanced metastatic renal-cell carcinoma: results of the combination of interleukin-2, alpha-interferon, 5-fluorouracil, vinblastine, and 13-cis-retinoic acid. World Journal of Urology, 13: 174-177, 1995.

38. Bonhommefaivre, L., Paule, B., Urien, S., Rudant, E., Bottius, L., Pradel, D., Marrot, D., All-trans retinoic acid, Hplc assay, Interferon alpha 2a, Pharmacokinetics, and Renal cell cancer pharmacokinetics of all-trans retinoic acid (ATAR) in patients with renal cancer concomitantly treated with interferon alpha 2a (IFN). International Journal of Pharmaceutics, 134: 99-104, 1996.

All references cited are incorporated herein by reference.

The compositions of this invention possess valuable pharmacological properties. They inhibit neoplasm cell proliferation and or angiogenesis in cancer therapy in human and veterinary medicine. Administration is contemplated to include chronic, acute or intermittent regimens.

The compositions are particularly useful in treating renal cancers and other solid tumors.

In addition, the compositions can be used in in vitro methodologies, including diagnostics or screening procedures (e.g., in an assay sensitive cancer types). In some embodiments, tissues, cells or material treated in vitro or extra corporeally will, thereafter, be reintroduced into a subject (which need not be the source of origin of the tissue, cells or material). Compounds of the present invention can be employed in admixture with carriers, excipients and other drugs, and radiation therapy.

The compositions of this invention are generally administered to animals, including but not limited to mammals such as livestock, household pets, humans, cattle, cats, dogs, poultry, etc.

The pharmacologically active compositions of this invention can be processed in accordance with conventional methods of Galenic pharmacy to produce medicinal agents for administration to patients, e.g., mammals including humans.

The compositions of this invention can be employed in admixture with conventional excipients, i.e., pharmaceutically acceptable organic or inorganic carrier substances suitable for parenteral, enteral (e.g., oral or inhalation) or topical application which do not deleteriously react with the active compositions. Suitable pharmaceutically acceptable carriers include but are not limited to water, salt solutions, etc. The pharmaceutical preparations can be sterilized and if desired mixed with auxiliary agents, e.g. They can also be combined where desired with other active agents, including radiation or other antineoplastic therapy.

In some embodiments of the present invention, dosage forms include instructions for the use of such compositions.

For parenteral application, particularly suitable are injectable, sterile solutions, preferably suspensions. Ampules are convenient unit dosages.

Sustained or directed release compositions can be formulated, e.g., liposomes or those wherein the active component is protected with differentially degradable coatings, e.g., by microencapsulation, multiple coatings, etc. It is also possible to freeze-dry the new compositions and use the lyophilizates obtained, for example, for the preparation of products for injection.

Generally, the two compositions of this invention are dispensed in unit dosage form comprising liposomal ATRA of from 15 to 300 or more mg/m² and particularly about 90 mg/m² ATRA. Interferon is administered at from about 1,000,000 to about 25,000,000 IU, and particularly from about 3,000,00 to about 5,000,000 sc and from daily to about 5 out of 7 days to about 3 out of 7 days per week.

It will be appreciated that the actual preferred amounts of active compositions in a specific case will vary according to the specific compositions being utilized, the particular compositions formulated, the mode of application, and the particular situs and organism being treated. Dosages for a given host can be determined using conventional considerations, e.g., by customary comparison of the differential activities of the subject compositions and of a known agent, e.g., by means of an appropriate, conventional pharmacological protocol.

1. A method of inhibiting the growth of cancer cells comprising exposing cancerous cells to a therapeutically effective amount of a composition which comprises at least one interferon and a retinoid, wherein said retinoid is associated with lipid carrier particles.

2. The method of claim 1 wherein the retinoid is retinoic acid.

3. The method of claim 2 wherein the retinoic acid is all-trans retinoic acid.

4. The method of claim 3 wherein lipid carrier particles comprise all-trans retinoic acid, lipid, and a triglyceride and the molar ratio of retinoid to lipid is at least about 15:85, where the triglyceride is at least about 15% by weight of the composition, and where the composition is stable in an aqueous environment.

5. The method of claim 1 comprising administering said retinoid composition in doses administered over a period of at least one-half hour.

6. The method of claim 1 comprising administering said retinoid composition at a frequency of about every other day or less frequent.

8. The method of claim 1 wherein the cancer is a renal cancer.

9. A method of inhibiting the growth of cancer cells comprising
exposing cancerous cells to a therapeutically effective amount of a composition which comprises at least one interferon and further
co-timely exposing of said cancerous cells to a therapeutically effective amount of a retinoid, wherein said retinoid is associated with lipid carrier particles.

10. A therapeutic treatment kit for the treatment of cancer comprising interferon, retinoid and instructional materials for the combined use of said retinoid and interferon.
